(19) United States
(12) Patent Application Publication
(10) Pub. No.: US 2019/0032231 A1
(43) Pub. Date: Jan. 31, 2019

(54) METAL-CONTAINING CLUSTER CATALYST, AND ELECTRODE FOR CARBON DIOXIDE REDUCTION AND CARBON DIOXIDE REDUCTION APPARATUS INCLUDING THE SAME

(71) Applicant: Furukawa Electric Co., Ltd., Tokyo (JP)

(72) Inventors: Yoshinori KAZAMA, Tokyo (JP); Hideki AIZAWA, Tokyo (JP); Toshio TANI, Tokyo (JP); Kojiro INAMORI, Tokyo (JP)

(73) Assignee: Furukawa Electric Co., Ltd., Tokyo (JP)

(21) Appl. No.: 16/144,800

(22) Filed: Sep. 27, 2018

Related U.S. Application Data
(63) Continuation of application No. PCT/JP2017/009885, filed on Mar. 13, 2017.

(30) Foreign Application Priority Data
Mar. 28, 2016 (JP) 2016-064611

(51) Int. Cl. C25B 11/04; C07C 1/12; C25B 3/04; C25B 9/10

(52) U.S. Cl. CPC C25B 11/0478; C07C 1/12; C07C 2523/72; C25B 9/10; C07C 2521/18; C25B 3/04

(57) ABSTRACT

A metal-containing cluster catalyst includes a cluster including at least one metal atom (M), a metal of each of the at least one metal atom (M) being selected from the group consisting of gold, silver, copper, platinum, rhodium, palladium, nickel, cobalt, iron, manganese, chromium, iridium, and ruthenium. The metal-containing cluster catalyst is used for reducing carbon dioxide.

### CROSS REFERENCE TO RELATED APPLICATIONS

This is a continuation application of International Patent Application No. PCT/JP2017/009885 filed Mar. 13, 2017, which claims the benefit of Japanese Patent Application No. 2016-064611, filed Mar. 28, 2016, the full contents of both of which are hereby incorporated by reference in their entirety.

### BACKGROUND

### Technical Field

The present disclosure relates to a metal-containing cluster catalyst, and also to an electrode for carbon dioxide reduction and a carbon dioxide reduction apparatus including the same.

### Background

Generally, a catalyst means a substance that changes the reaction rate of a substance system causing a chemical reaction but itself is not chemically changed, and selectivity and reaction efficiency with respect to a specific chemical reaction differ depending on the kind (material, morphology or the like) of the catalyst.

Metal materials are widely used as catalyst materials, and noble metal materials are particularly of importance due to their good reactivity. For example, Japanese Patent Application Laid-Open No. 2007-090164 discloses a noble metal catalyst having selectivity in a specific reaction. In recent years, attention is also being focused on oxide catalysts. Japanese Patent Application Laid-Open No. 2007-301470 discloses an oxide catalyst having excellent catalytic activity and selectivity.

However, in the reduction reaction of carbon dioxide, the control of the chemical reaction with selectivity is not yet sufficient, and it is not possible to obtain the product at a high reaction efficiency. Therefore, development of a catalyst capable of controlling the reduction reaction of carbon dioxide with high selectivity is desired.

The present disclosure is related to providing a metal-containing cluster catalyst capable of accelerating/controlling the reduction reaction of carbon dioxide with high catalytic activity and selectivity, and an electrode for carbon dioxide reduction and a carbon dioxide reduction apparatus including the same.

### SUMMARY

According to a first aspect of the present disclosure, a metal-containing cluster catalyst includes a cluster including at least one metal atom (M), a metal of each of the at least one metal atom (M) being selected from the group consisting of gold, silver, copper, platinum, rhodium, palladium, nickel, cobalt, iron, manganese, chromium, iridium, and ruthenium, wherein the metal-containing cluster catalyst is used for reducing carbon dioxide.

According to a second aspect of the present disclosure, an electrode for carbon dioxide reduction includes a metal-containing cluster catalyst, the metal-containing cluster catalyst including a cluster including at least one metal atom (M), a metal of each of the at least one metal atom (M) being selected from the group consisting of gold, silver, copper, platinum, rhodium, palladium, nickel, cobalt, iron, manganese, chromium, iridium, and ruthenium, wherein the metal-containing cluster catalyst is used for reducing carbon dioxide.

According to a third aspect of the present disclosure, a carbon dioxide reduction apparatus includes an electrode for carbon dioxide reduction, the electrode for carbon dioxide reduction including a metal-containing cluster catalyst including a cluster including at least one metal atom (M), a metal of each of the at least one metal atom (M) being selected from the group consisting of gold, silver, copper, platinum, rhodium, palladium, nickel, cobalt, iron, manganese, chromium, iridium, and ruthenium, wherein the metal-containing cluster catalyst is used for reducing carbon dioxide.

The metal-containing cluster catalyst of the present disclosure exhibits excellent performance in the reduction reaction of carbon dioxide.

### BRIEF DESCRIPTION OF THE DRAWINGS

FIG. 1 is a block diagram showing the configuration of an electrolysis apparatus 1.

FIG. 2A is a schematic diagram showing the configuration of an electrolysis cell 3, and

FIG. 2B is a diagram showing the configuration of an electrolysis cell 3a.

FIG. 3 is a general schematic diagram showing an electrode producing apparatus 27 used in producing an electrode including a copper porous body according to Comparative Examples of the present disclosure.

FIG. 4 is a schematic diagram showing a reduction testing apparatus 50 when a reduction test is performed on Examples.

FIG. 5 is an enlarged schematic diagram showing an electrolysis cell 53 portion (H portion) of the reduction testing apparatus 50 of FIG. 4.

### DETAILED DESCRIPTION

Hereinafter, embodiments of a metal-containing cluster catalyst according to the present disclosure, and an electrode for carbon dioxide reduction and a carbon dioxide reduction apparatus including the same will be described in detail.

The metal-containing cluster catalyst according to the present embodiment is a cluster including at least one metal atom (M), a metal of each of the least one metal atom (M) being selected from the group consisting of gold (Au), silver (Ag), copper (Cu), platinum (Pt), rhodium (Rh), palladium (Pd), nickel (Ni), cobalt (Co), iron (Fe), manganese (Mn), chromium (Cr), iridium (Ir), and ruthenium (Ru). Here, the term “cluster” refers to an atomic aggregate wherein a plurality of atoms are bonded.

Such metal-containing cluster catalyst exhibits excellent performance in reduction reaction of carbon dioxide, and thus is suitably used as a catalyst for reducing carbon dioxide.

The metal atom (M) contained in the cluster according to the present embodiment is of a metal selected from Au, Ag, Cu, Pt, Rh, Pd, Ni, Co, Fe, Mn, Cr, Ir, and Ru. The cluster (hereinafter, referred to as “metal-containing cluster”) exhibits excellent performance in the reduction reaction of carbon dioxide. Of these, taking the excellent reduction performance into consideration, the metal atom (M) is preferably of a metal selected from Cu, Ag, Pd, and Au. Particularly, taking into consideration that a hydrocarbon (methane, ethylene or the like) can be selectively generated in the reduction reaction of carbon dioxide, the metal atom (M) is more preferably of Cu.

The metal-containing cluster is not particularly limited as long as the metal-containing cluster contains the metal atoms (M) described above. The metal-containing cluster may be a cluster composed of a simple substance of the metal atom (M), an alloy containing the metal atom (M), a metal oxide containing the metal atom (M), or a composite oxide containing the metal atom (M). The alloy or composite oxide containing the metal atom (M) may be an alloy or composite oxide containing at least one metal atom, a metal of each of the at least one metal being selected from the group consisting of Au, Ag, Cu, Pt, Rh, Pd, Ni, Co, Fe, Mn, Cr, Ir, and Ru. The alloy or composite oxide containing the metal atom (M) may be an alloy or composite oxide containing metal atoms of two or more metals selected from the above, or an alloy or composite oxide containing a metal atom other than the above capable of forming an alloy or a composite with the metal atom (M). Particularly, the metal-containing cluster is preferably composed of a metal oxide containing the metal atom (M).

The metal-containing cluster is preferably composed of a simple substance of the metal atom (M) or a metal oxide of the metal atom (M) and represented by the following general formula (1):

MₙOₘ (1),

where

M represents the aforementioned metal atom (M), and O represents an oxygen atom, and

n and m are integers.

Further, in the aforementioned formula (1), n is preferably 30 or less, and more preferably 15 or less. n is preferably 6 or more, and more preferably 9 or more. With the aforementioned range, the size of the cluster can be controlled, and a degree of oxidation (valence) can also be controlled.

In the aforementioned formula (1), m is, with respect to n, such that a ratio m/n is preferably 0 to 2, more preferably 0.5 to 1.5, and still more preferably 0.55 to 0.75. The aforementioned range provides a degree of oxidation specific to the cluster, and thus catalyst performance improves. In formula (1), when m/n is 0, the metal-containing cluster is composed of a simple substance of the metal atom (M).

A primary particle diameter of the metal-containing cluster is preferably 0.1 to 3.0 nm, more preferably 0.5 to 2.0 nm, and still more preferably 0.6 to 1.2 nm. The aforementioned range provides an increase in the total surface area of particles of the metal-containing cluster and a degree of oxidation specific to the cluster, and thus catalyst performance improves. The primary particle diameter is measured using mass spectrometry (MS), a transmission electron microscope (TEM), a dynamic light scattering method (DLS) or the like.

The metal-containing cluster can be produced in a liquid phase or a gas phase by a known method. Examples of a method for producing the metal-containing cluster in the liquid phase include a method using a dendrimer. Examples of a method for producing the metal-containing cluster in the gas phase include an ion sputtering method, a plasma discharge method, and a laser vaporization method (laser ablation). Of these, in terms of efficiently producing a metal-containing cluster containing a metal atom (M) having a special valence, it is preferable to produce the metal-containing cluster by a method using laser ablation or a dendrimer.

Laser ablation is a phenomenon in which clusters scatter when a solid surface is irradiated with strong laser light and a surface layer that has locally come to an increased temperature evaporates. The laser ablation may also be referred to as laser sputtering. An apparatus performing laser ablation is not particularly limited, and laser ablation can be performed by using a known apparatus.

The method using a dendrimer is not particularly limited, and can be performed by using a known method. For example, a metal cluster can be produced by, mixing a solution containing a dendrimer and a solution containing a metal compound corresponding to an intended metal-containing cluster to synthesize a metal complex of the dendrimer, and performing: (1) a method in which the metal complex is reduced by reduction using a reducing agent such as sodium borohydride, electrochemical reduction, photochemical reduction or the like, to deposit a metal on the dendrimer; (2) a method in which a solution containing the dendrimer metal complex is heated to remove a solvent, to thereby deposit a metal or a salt thereof on the dendrimer; or the like. Examples of the dendrimer include a polyamideamine (PAMAM) dendrimer, a polypropyleneimine (PPI) dendrimer, and a phenylazomethine dendrimer (DPA). Examples of the metal compound include a chloride and nitrate of the metal corresponding to the intended metal-containing cluster. For example, known solvents such as water and alcohol may be used as the solvent.

Furthermore, a carbon material encapsulating the metal-containing cluster is obtained by firing the metal-containing cluster obtained as described above. The firing can be, in the case of a high temperature (for example, 400° C. or above), performed in an inactive gas atmosphere such as nitrogen or a rare gas including argon, and, in the case of a comparatively low temperature (for example, 200° C. or above), can be performed in the air. After the above firing, a heat treatment can also be performed in a reduction atmosphere such as hydrogen.

The form of use of the metal-containing cluster catalyst according to the present embodiment is not particularly limited, and the metal-containing cluster catalyst is preferably used as a composite material with the metal-containing cluster catalyst supported on a carrier.

Examples of the carrier include, but are not particularly limited to, carbon, a metal, a semiconductor, ceramics, and a polymer. The carrier may be appropriately selected depending on the application, use environment or the like of the composite material. Specifically, when the composite material is used as a conductive material, the carrier is preferably carbon or a metal. When the composite material is used as a photocatalyst, the carrier is preferably a semiconductor.

The polymer suitably used as the carrier is, for example, a chainlike polymer, a single polymer or the like. Examples of the chainlike polymer include polyvinyl pyrrolidone (PVP), polyethylene glycol (PEG), and polyvinylidene fluoride (PVdF). Examples of the single polymer include ferritin, thiolate, phosphine, and alkine.

The metal-containing cluster catalyst according to the present embodiment can be suitably used as a material of the electrode for carbon dioxide reduction. Therefore, the electrode for carbon dioxide reduction according to the present embodiment preferably contains the above metal-containing cluster catalyst. A method for forming the electrode is not particularly limited, and can be performed by a known method.

Specifically, for example, an electrode including the metal-containing cluster catalyst can be produced by dispersing the aforementioned carbon material encapsulating the metal-containing cluster using the dendrimer in a solvent to obtain a dispersion liquid, mixing the dispersion liquid with a binder to obtain a mixture, mixing the mixture with a conductive material if needed to obtain a coating material, and directly applying the coating material to a current collector, an ion-exchange membrane or the like, followed by drying.

Examples of the binder include, but are not particularly limited to, polyvinylidene fluoride (PVdF), a mixture of carboxymethylcellulose sodium (CMC) and styrene butadiene copolymer (SBR), and polytetrafluoroethylene (PTFE).

The electrode including the metal-containing cluster catalyst according to the present disclosure can be suitably used as a cathode electrode of the carbon dioxide reduction apparatus to be described later.

The carbon dioxide reduction apparatus according to the present embodiment preferably include the aforementioned electrode for carbon dioxide reduction. A method for producing the apparatus is not particularly limited, and can be performed by a known method.

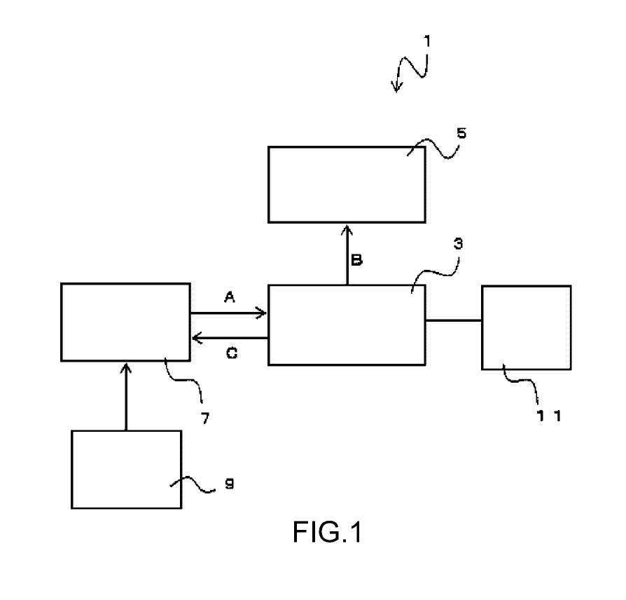

Hereinafter, an electrolysis apparatus 1 shown in FIG. 1 will be described as an example of the carbon dioxide reduction apparatus according to the present embodiment. In FIG. 1, the electrolysis apparatus 1 mainly includes an electrolysis cell 3, a gas recovery apparatus 5, an electrolysis solution circulation apparatus 7, a carbon dioxide supply part 9, and a power supply 11. Needless to say, the configuration of the carbon dioxide reduction apparatus according to the present embodiment is not limited to the configuration shown in FIG. 1, and the carbon dioxide reduction apparatus may be appropriately modified and used if needed.

The electrolysis cell 3 is a portion that reduces the target substance, and in the present embodiment, particularly a portion that reduces carbon dioxide (also includes the case where it is a carbonate ion or carbonate in a solution. Hereinafter, merely referred to as carbon dioxide or the like). The electrolysis cell 3 is supplied with electric power from the power supply 11. The electrolysis cell 3 will be described in detail later.

The electrolysis solution circulation apparatus 7 is a portion that circulates a cathode side electrolysis solution to the cathode electrode of the electrolysis cell 3.

The carbon dioxide supply part 9 is a tank or the like that stores carbon dioxide, for example, and can hold carbon dioxide and supply a predetermined amount of carbon dioxide to the electrolysis solution circulation apparatus 7. The carbon dioxide supply part 9 can also hold a solution already in the form of a carbonate ion, carbonate or the like in place of the carbon dioxide, and supply a predetermined amount of the solution to the electrolysis solution circulation apparatus 7.

The gas recovery apparatus 5 is a portion that recovers a gas generated by reduction by the electrolysis cell 3. The gas recovery apparatus 5 is capable of collecting a gas such as a hydrocarbon generated at the cathode electrode of the electrolysis cell 3. The gas recovery apparatus 5 may allow the gas to be separated into each type of gas.

The electrolysis apparatus functions as follows. As described above, an electrolysis potential from the power supply is applied to the electrolysis cell. The electrolysis solution is supplied to the cathode electrode of the electrolysis cell by the electrolysis solution circulation apparatus. At the cathode electrode of the electrolysis cell, carbon dioxide or the like in the supplied electrolysis solution is reduced. When the carbon dioxide or the like is reduced, a hydrocarbon such as ethane or ethylene is mainly generated.

The hydrocarbon gas generated at the cathode electrode is recovered by the gas recovery apparatus. In the gas recovery apparatus, the gas can be separated and stored if needed.

As a result of carbon dioxide or the like being reduced and consumed at the cathode electrode, the concentration of carbon dioxide or the like in the electrolysis solution decreases. Carbon dioxide or the like decreased due to the reduction reaction is always refilled, and the concentration of carbon dioxide is always maintained in a predetermined range. Specifically, a part of the electrolysis solution is recovered by the electrolysis solution circulation apparatus, and an electrolysis solution having a predetermined concentration is always supplied. As described above, the hydrocarbon can be always generated under a given condition at the electrolysis cell 3.

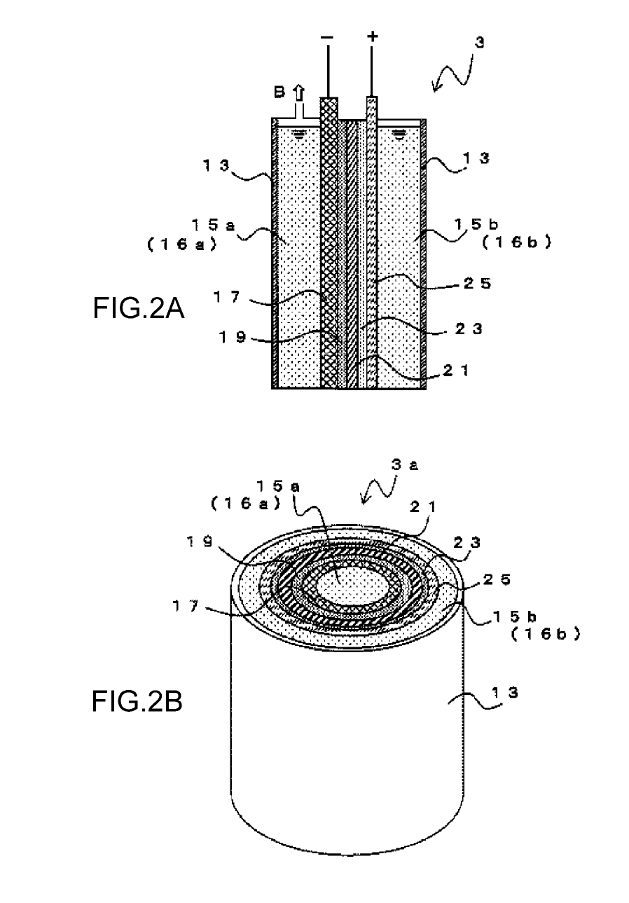

The electrolysis cell 3 will now be described in detail. FIG. 2A is a diagram showing an example of the configuration of the electrolysis cell 3. The electrolysis cell 3 mainly includes a tank 16a as a cathode tank, a metal mesh 17, a cathode electrode 19, an ion-exchange membrane 21, an electrolyte 23, an anode electrode 25, and a tank 16b as an anode tank. Needless to say, the configuration of an electric cell of the carbon dioxide reduction apparatus according to the present embodiment is not limited to the configuration shown in FIG. 2A, and the configuration of the electrolysis cell may be appropriately modified and used if needed.

Electrolysis solutions 15a and 15b are respectively held in the cathode tank 16a and the anode tank 16b. A hole for recovering a generated gas is formed in the upper part of the tank 16a on the side of the cathode electrode, and is connected to the gas recovery apparatus, not shown. That is, the gas generated at the cathode electrode is recovered from the hole. A pipe or the like is connected to the cathode tank 16a, and is connected to the electrolysis solution circulation apparatus 7, not shown. That is, the electrolysis solution 15a in the cathode tank 16a can be always circulated by the electrolysis solution circulation apparatus 7. The circulation of the electrolysis solution on the side of the anode tank 16b may also be similarly allowed if needed.

The electrolysis solution 15a as a cathode electrolysis solution is preferably an electrolysis solution capable of dissolving a large amount of carbon dioxide or the like. For example, an alkaline solution such as a sodium hydroxide aqueous solution or a potassium hydroxide aqueous solution, monomethanolamine, methylamine, other liquid amine, or a mixed solution of the liquid amine and electrolyte aqueous solution, or the like is used. Acetonitrile, benzonitrile, methylene chloride, tetrahydrofuran, propylene carbonate, dimethylformamide, dimethyl sulfoxide, methanol, ethanol or the like may be used.

The electrolyte aqueous solution is not particularly limited, and a potassium chloride aqueous solution, a sodium chloride aqueous solution, a sodium hydroxide aqueous solution, a potassium hydroxide aqueous solution, a sodium hydrogen carbonate aqueous solution, a potassium carbonate aqueous solution or the like may be used, for example.

The electrolysis solution 15b as an anode electrolysis solution is not particularly limited, and a potassium chloride aqueous solution, a sodium chloride aqueous solution, a sodium hydrogen carbonate aqueous solution, a potassium hydrogen carbonate aqueous solution or the like may be used, for example.

The metal mesh 17 is a member connected to the anode electrode side of the power supply and applies an electric current to the cathode electrode 19. The metal mesh 17 is, for example, a copper mesh or a stainless steel mesh, and for example, a stainless steel SUS304 400 mesh (25 μm in thickness, manufactured by Nilaco Corporation) may be used.

The ion-exchange membrane 21 is not particularly limited, and, for example, a hydrocarbon-based, perfluorocarbon-based ion-exchange membranes or the like may be used. The ion-exchange membrane 21 is particularly desirably a negative ion exchange membrane, and a Nafion membrane, a polyvinylidene fluoride (PVDF) membrane or the like may be used. For example, “SELEMION (registered trademark) AMV” manufactured by Asahi Glass Co., Ltd. may be used. The ion-exchange membrane 21 is used when manufacturing the cathode electrode 19 to be described later, and exhibits a function as the support member of the metal-containing cluster catalyst constituting the cathode electrode 19. The use of the ion-exchange membrane as a support member provides a simple configuration of a reduction part during electrolysis described later.

The electrolyte 23 is provided if needed. The electrolyte 23 interposed between the ion-exchange membrane 21 and the anode electrode 25 to be described later is not particularly limited, and a polymer electrolyte such as polyvinylidene fluoride, polyacrylic acid, polyethylene oxide, polyacrylonitrile, or polymethyl methacrylate, or a potassium chloride aqueous solution, a sodium chloride aqueous solution or the like may be used.

The anode electrode 25 is connected to a positive electrode of the power supply. The anode electrode 25 is not particularly limited, and, for example, titanium, platinum, titanium coated with platinum (Ti/Pt), stainless steel, copper, carbon or the like may be used. Ti/Pt is particularly preferable since deterioration is less. The shape to be used is not particularly limited, and may be a plate shape, a punching metal shape, a mesh shape, or a nonwoven fabric shape. In terms of reducing the thickness of the electrolysis cell, and allowing the use even if the electrolysis cell has a curved shape, the nonwoven fabric shape is preferable.

An electrode including the metal-containing cluster catalyst according to the present disclosure is used as the cathode electrode 19. The use of the electrode including the metal-containing cluster catalyst according to the present disclosure as the cathode electrode 19 makes it possible to increase the amount of carbon dioxide reduced and to selectively control the reduction reaction of the carbon dioxide. Reduction efficiency can also be improved in respect of selectivity.

As the electrolysis cell according to the present embodiment, an electrolysis cell 3a as shown in FIG. 2B may also be used. The electrolysis cell 3a has a configuration substantially similar to that of the electrolysis cell 3, but contrary to the electrolysis cell 3 in which elements each having a plate shape are stacked, each elements are sequentially arranged substantially concentrically from the center to the outer periphery in a radial direction. Since each of the elements constituting the electrolysis cell 3a is similar to each of the elements constituting the electrolysis cell 3, like description will be omitted.

Hereinbefore, embodiments of the present disclosure have been described. However, the present disclosure is not limited to the above embodiments, and includes all aspects included in the concept of the present disclosure and appended claims, and various modifications can be made within the scope of the present disclosure.

### EXAMPLES

Hereinafter, Examples and Comparative Examples will be described to further clarify the advantageous effects of the present disclosure. However, the present disclosure is not limited to these Examples.

### Example 1

First, a chloroform solution of a phenylazomethine dendrimer and an acetone solution of copper chloride of 12 molar equivalents with respect to the dendrimer were mixed to synthesize a phenylazomethine dendrimer copper complex. Then, to this solution, an excessive amount of sodium borohydride was added to reduce the phenylazomethine dendrimer copper complex, and a phenylazomethine dendrimer copper cluster was synthesized. The synthesized phenylazomethine dendrimer copper cluster was fired in a nitrogen atmosphere to synthesize a carbon material encapsulating a copper-containing cluster.

Furthermore, the obtained carbon material enxapsulating a copper-containing cluster was dispersed in N-methyl-2-pyrrolidone (NMP) to obtain a dispersion liquid, and polyvinylidene fluoride (PVDF) as a binder was added to the dispersion liquid, followed by mixing to obtain a coating material. The coating material was applied onto an ion-exchange membrane, followed by drying, to produce an electrode including a copper-containing cluster catalyst.

### Example 2

In Example 2, a carbon material encapsulating a copper-containing cluster, and an electrode including a copper-containing cluster catalyst including such carbon material were produced by a method similar to that of Example 1 except that the firing atmosphere of a phenylazomethine dendrimer copper cluster was air atmosphere in place of the nitrogen atmosphere, and the firing temperature was lower than that of Example 1.

### Example 3

In Example 3, a carbon material encapsulating a copper-containing cluster, and an electrode including a copper-containing cluster catalyst including such carbon material were produced by a method similar to that of Example 1 except that the firing temperature of a phenylazomethine dendrimer copper cluster was lower than that in Example 1.

### Example 4

In Example 4, a carbon material encapsulating a copper-containing cluster, and an electrode including a copper-containing cluster catalyst including such carbon material were produced by a method similar to that of Example 1 except that a phenylazomethine dendrimer copper cluster was fired in a nitrogen atmosphere, and then further fired in a hydrogen atmosphere.

### Example 5

In Example 5, a carbon material encapsulating a silver-containing cluster, and an electrode including a silver-containing cluster catalyst including such carbon material were produced by a method similar to that of Example 1 except that a phenylazomethine dendrimer silver complex was synthesized by mixing a chloroform solution of a phenylazomethine dendrimer with an acetone solution of silver nitrate of 12 molar equivalents with respect to the dendrimer.

### Comparative Example 1

In Comparative Example 1, with a method to be shown later, copper was deposited on an ion-exchange membrane by an electroless plating method to obtain an electrode having a copper porous body in which the deposited copper particles are aggregated.

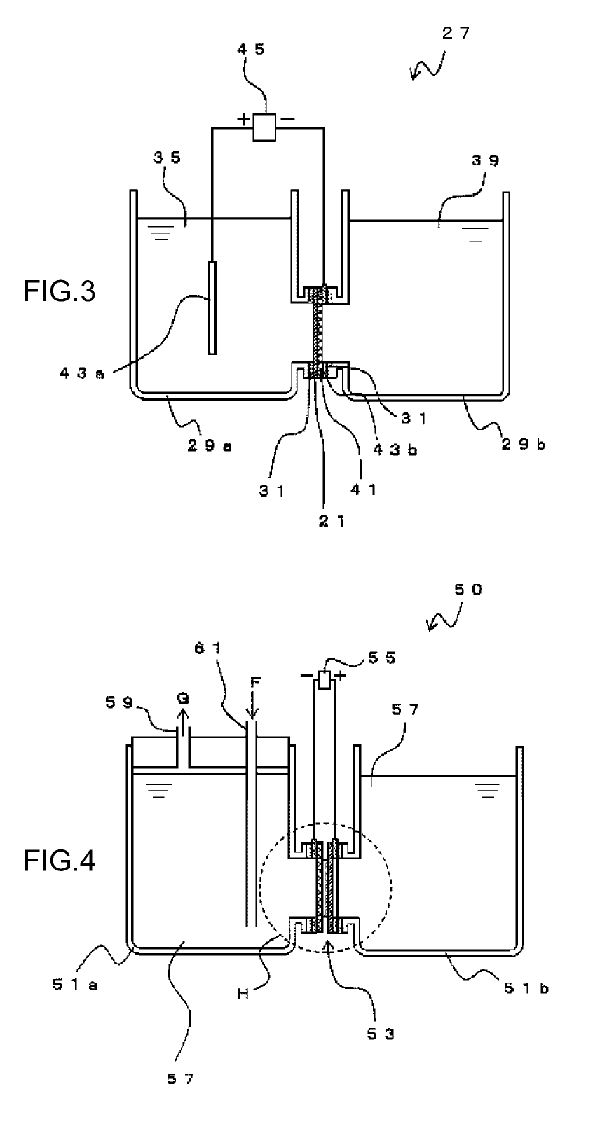

First, 30 mL of a copper acetate aqueous solution of 5 mmol/L was placed in a tank 29a of an electrode producing apparatus 27 shown in FIG. 3, and a mixed solution of 142 μL of a sodium hydroxide solution of 12% by mass (NaOH of 14 mol/L manufactured by Aldrich) and 29.858 mL of distilled water was placed in a tank 29b. The electrode producing apparatus 27 having such a configuration was allowed to stand at room temperature for 1 hour, and copper was deposited on the ion-exchange membrane 21 by an electroless plating method, to obtain an electrode having a copper porous body.

### Comparative Example 2

In Comparative Example 2, an electrode having a copper porous body was obtained by a method similar to that of Comparative Example 1 except that the electrode produced in Comparative Example 1 was fired in an air atmosphere.

### Comparative Example 3

In Comparative Example 3, an electrode having a copper porous body was obtained by a method similar to that of Comparative Example 1 except that the electrode produced in Comparative Example 1 was fired in a nitrogen atmosphere.

### Comparative Example 4

In Comparative Example 4, a method similar to that of Comparative Example 1 was performed except that 30 mL of a copper acetate aqueous solution of 5 mmol/L of Comparative Example 1 was changed to 30 mL of a silver nitrate aqueous solution of 5 mmol/L, and an electrode having a silver porous body was produced.

### [Evaluation]

Characteristics to be shown later were evaluated for the above catalysts of Examples and Comparative Examples. Evaluation conditions of each of the characteristics are as follows. The results are shown in Table 1.

### [1] Composition and Valence of Metal (M)

Composition analysis and valence evaluation were performed for the above metal-containing cluster and metal-containing porous body constituting the catalyst using an inductively-coupled plasma sample analysis method and X-ray photoemission spectroscopy.

As a sample for the analysis, in each of Examples 1 to 4, a carbon material containing a copper-containing cluster before producing the electrode was used, and in Example 5, the carbon material containing a silver-containing cluster before producing the electrode was used. In each of Comparative Examples 1 to 4, a metal material obtained by scraping and separating the porous body part on the ion-exchange membrane was used.

### [2] Primary Particle Diameter

For each of Examples 1 to 5, particles of the carbon material containing a metal-containing cluster before producing the electrode, and for each of Comparative Examples 1 to 4, particles of the metal-containing porous body obtained from the ion-exchange membrane were photographed at a magnification capable of clearly recognizing the outline of a primary particle (independent particle not aggregated with other particle) using a transmission electron microscope (TEM, manufactured by JEOL Co., Ltd.), and the following analysis was performed for each of Examples and Comparative Examples. First, 100 particles (primary particles) were selected at random from the photographed image, and a projected area for each particle was obtained by an image processing apparatus. The total occupied area of the particles was calculated from the total of the projected areas. An average occupied area per particle was calculated by dividing the total occupied area with the number (100 particles) of the selected particles, and the diameter of the circle equivalent to the area (average circle equivalent diameter per particle) was obtained as a primary particle diameter.

### [3] Reduction Test: Gas Concentration and Current Efficiency

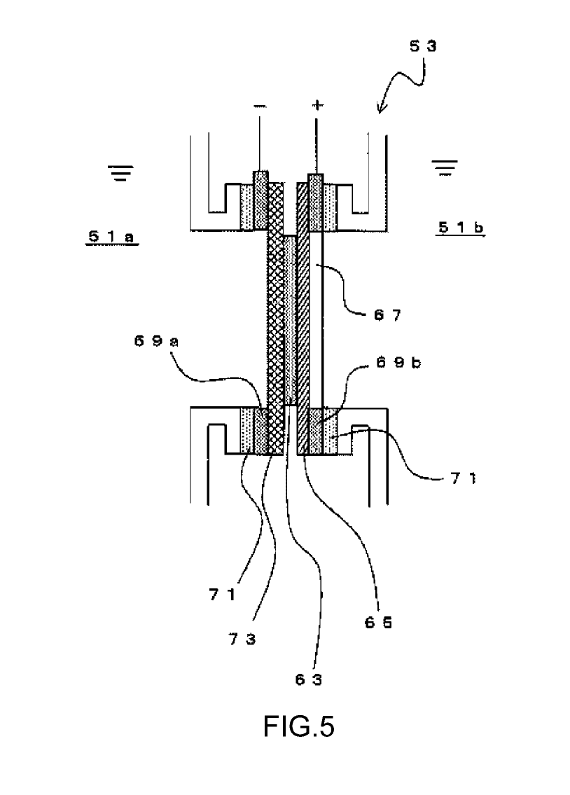

A reduction test was carried out using a carbon dioxide reduction testing apparatus 50 shown in FIG. 4. Here, the above electrodes according to Examples and Comparative Examples were used as a cathode electrode 69a. FIG. 5 is a diagram showing an electrolysis cell 53, and is an enlarged view of an H portion in FIG. 4. The reduction testing apparatus 50 mainly includes a first tank 51a, a second tank 51b, an electrolysis cell 53, a power supply 55, an analysis tube 59, and a supply tube 61.

The two tanks 51a and 51b are divided by the electrolysis cell 53. Sodium hydrogen carbonate solution 57 is placed in each of the first tank 51a and the second tank 51b. A sodium hydrogen carbonate solution of 50 mmol/L was used as the sodium hydrogen carbonate solution 57, and 30 mL of a solution was used for each tank. The upper part of the first tank 51a is sealed with a lid. The supply tube 61 and the analysis tube 59 are provided so as to pass through the lid. The supply tube 61 is connected to a carbon dioxide supply source, not shown, and an end portion of the supply tube 61 is immersed in the sodium hydrogen carbonate solution 57. The end portion of the supply tube 61 extends to the vicinity of the lower base part of the first tank 51a. The sodium hydrogen carbonate solution 57 in the first tank 51a is always agitated by supplying carbon dioxide from the supply tube 61 so that the concentration of the sodium hydrogen carbonate solution 57 is substantially maintained at a given value. Therefore, the same effect as that of the circulation of the sodium hydrogen carbonate solution 57 in the first tank 51a can be obtained.

The end portion of the analysis tube 59 passes through the lid part, and is arranged in a gas part between the lid part and the surface of the solution without being contact with the sodium hydrogen carbonate solution 57. That is, the analysis tube 59 is capable of collect a generated gas or the like. The analysis tube 59 is connected to a gas analysis apparatus, not shown, and the collected gas is guided to the analysis apparatus.

As shown in FIG. 5, in the electrolysis cell 53, a copper-containing cluster catalyst 63 (This is for the case of the electrode in Examples. In the case of the electrode in Comparative Examples, it is the copper porous body 63. The same applies hereinafter.) as a cathode electrode is formed on an ion-exchange membrane 65. A metal mesh 73 is provided such that the copper-containing cluster catalyst 63 is sandwiched between the metal mesh 73 and the ion-exchange membrane 65. That is, the metal mesh, the copper-containing cluster catalyst, and the ion-exchange membrane are arranged in turn from the side of the first tank 51a, and sandwiched between the cathode electrode 69a and an anode electrode 69b. Furthermore, the cathode electrode 69a and the anode electrode 69b are sandwiched by a sealing member 71 from the outside, and are fixed by a clamp or the like, not shown.

Here, a rubber packing was used as the sealing member 71. The cathode electrode 69a is a member applying an electric current to the metal mesh 73, and a ring-shaped Ti/Pt electrode was used. The metal mesh 73 is a copper mesh. The metal mesh 73 electrically contacts with the copper-containing cluster catalyst 63, and functions as a cathode itself. As the copper mesh, “copper 100 mesh metal gauze” (0.11 mm in thickness, manufactured by Nilaco Corporation) was used. The copper-containing cluster catalyst 63 is the above electrode produced in Examples (or Comparative Examples). As the ion-exchange membrane 65, “SELEMION (registered trademark) AMV” manufactured by Asahi Glass Co., Ltd. was used.

As the anode electrode 69b, a ring-shaped Ti/Pt electrode holding a metallic nonwoven fabric 67 as the anode electrode, and electrically contacting with the metallic nonwoven fabric 67 was used. A nonwoven fabric made of Pt was used as the metallic nonwoven fabric 67. That is, the metallic nonwoven fabric 67 is held in a ring of the ring-shaped anode electrode 69b.

As shown in FIG. 4, the cathode electrode 69a and the anode electrode 69b are connected to the power supply 55. In the reduction test, electrolysis was performed at a current value of 2 mA and a voltage of 2.8 V for 60 minutes with the cathode electrode 69a as the cathode and the anode electrode 69b side as the anode.

Here, a carbon dioxide gas was bubbled at 10 mL/min from the supply tube 61 into a tank 51a on the side of the metal mesh 73 and copper-containing cluster catalyst 63 (the opposite side of the ion-exchange membrane 65) (arrow F direction in the figure). The gas generated from the cathode was collected with the analysis tube 59 (arrow G direction in the figure), and analyzed by gas chromatography. SUPELCO CARBOXEN 1010PLOT 30 m×032 mm ID was used as a column, and FID was used as a detector.

For the reactions in the cathode electrode, generation of methane, ethylene, and ethane shown below was closely studied.

CO₂+8H⁺+8e⁻→CH₄+2H₂O

2CO₂+12H⁺+12e⁻→C₂H₄+4H₂O

2CO₂+14H⁺+14e⁻→C₂H₆+4H₂O

The reaction at the anode electrode is as follows.

2H₂O4H⁺+4e⁻+O₂

Furthermore, current efficiency (Faraday efficiency) was calculated based on the amount of gas of the obtained product and the input current.

TABLE 1

| | Catalyst | | | | | | | | | |
|---|---|---|---|---|---|---|---|---|---|---|
| | | Contained | | Valence | Primary particle | Evaluation of | Product | | | |
| | Morphology | metal | Composition | of metal | diameter [nm] | characteristics | CH₄ | C₂H₄ | C₂H₆ | total |
| Example 1 | Cluster | Copper | Cu₁₂O₈ | +1.3 | 1.0 | Gas concentration [ppm] | 326 | 240 | 68 | 634 |
| | | | | | | Current efficiency [%] | 28.5 | 31.4 | 10.5 | — |
| Example 2 | Cluster | Copper | Cu₁₂O₁₂ | +2.0 | 1.2 | Gas concentration [ppm] | 251 | 193 | 54 | 498 |
| | | | | | | Current efficiency [%] | 21.9 | 25.2 | 8.3 | — |
| Example 3 | Cluster | Copper | Cu₁₂O₆ | +1.0 | 0.9 | Gas concentration [ppm] | 267 | 204 | 58 | 529 |
| | | | | | | Current efficiency [%] | 23.3 | 26.7 | 9.0 | — |
| Example 4 | Cluster | Copper | Cu₁₂ | +0 | 0.9 | Gas concentration [ppm] | 197 | 159 | 35 | 391 |
| | | | | | | Current efficiency [%] | 17.1 | 20.8 | 5.4 | — |
| Comparative | Porous body | Copper | Cu | +0 | 185 | Gas concentration [ppm] | 151 | 124 | 31 | 306 |
| Example 1 | | | | | | Current efficiency [%] | 13.2 | 16.2 | 4.8 | — |
| Comparative | Porous body | Copper | CuO | +2.0 | 185 | Gas concentration [ppm] | 162 | 130 | 45 | 337 |
| Example 2 | | | | | | Current efficiency [%] | 14.7 | 16.6 | 6.8 | — |
| Comparative | Porous body | Copper | Cu₂O | +1.0 | 185 | Gas concentration [ppm] | 170 | 144 | 55 | 369 |
| Example 3 | | | | | | Current efficiency [%] | 15.0 | 17.0 | 7.3 | — |
| Example 5 | Cluster | Silver | Ag₁₂ | +0 | 0.9 | Gas concentration [ppm] | 70 | 20 | 9.8 | 99.8 |
| | | | | | | Current efficiency [%] | 6.1 | 2.6 | 1.3 | — |
| Comparative | Porous body | Silver | Ag | +0 | 85 | Gas concentration [ppm] | 40 | 8.4 | 0 | 48.4 |
| Example 4 | | | | | | Current efficiency [%] | 3.5 | 1.1 | 0 | — |

As shown in Table 1, it was confirmed that each of the metal-containing cluster catalysts (Examples 1 to 5) has excellent catalytic activity than that of each of the porous body catalysts (Comparative Examples 1 to 4).

Specifically, it was confirmed that the amount of the product obtained by the reduction of carbon dioxide in the copper-containing cluster catalyst according to each of Examples 1 to 4 is more than the amount of the product obtained by the porous body catalyst according to each of Comparative Examples 1 to 3 similarly using copper, and the copper-containing cluster catalyst according to each of Examples 1 to 4 has excellent catalytic activity.

It was confirmed that the amount of the product obtained by the reduction of carbon dioxide in the silver-containing cluster catalyst according to Example 5 is more than the amount of the product obtained by the porous body catalyst according to Comparative Example 4 similarly using silver, and the silver-containing cluster catalyst according to Example 5 has excellent catalytic activity.

It was confirmed that the amount of a hydrocarbon (methane, ethylene, and ethane) obtained by the reduction reaction of carbon dioxide in each of the copper-containing cluster catalysts (Examples 1 to 4) is more than that in the silver-containing cluster catalyst (Example 5), and each of the copper-containing cluster catalysts has excellent hydrocarbon selectivity.

### Examples 6 to 27

In each of Examples 6 to 27, a carbon material encapsulating a copper-containing cluster, and an electrode including a copper-containing cluster catalyst using the carbon material were produced by a method similar to that of Example 1 except that the number of generations of a phenylazomethine dendrimer, the equivalent amounts of raw materials, the firing condition of a phenylazomethine dendrimer copper cluster, and the like were appropriately changed, and evaluated in a similar manner as with Example 1. The results are shown in Table 2. In Table 2, Examples 1 to 4 are similar to those shown in Table 1.

TABLE 2

| | Metal-containing cluster catalyst: CuₙOₘ | | | | | | | | | |
|---|---|---|---|---|---|---|---|---|---|---|
| | | | | | Primary | | | | | |
| | | | | Valence of | particle | Evaluation of | Product | | | |
| | n | m | m/n | Cu | diameter [nm] | characteristics | CH₄ | C₂H₄ | C₂H₆ | total |
| Example 6 | 3 | 2 | 0.67 | +1.3 | 0.60 | Gas concentration [ppm] | 301 | 228 | 60 | 589 |
| | | | | | | Current efficiency [%] | 26.3 | 29.8 | 9.3 | — |
| Example 7 | 4 | 3 | 0.75 | +1.5 | 0.60 | Gas concentration [ppm] | 272 | 204 | 57 | 533 |
| | | | | | | Current efficiency [%] | 23.8 | 26.7 | 8.8 | — |
| Example 8 | 5 | 3 | 0.60 | +1.2 | 0.75 | Gas concentration [ppm] | 279 | 202 | 54 | 535 |
| | | | | | | Current efficiency [%] | 24.4 | 26.4 | 8.4 | — |
| Example 9 | 6 | 4 | 0.67 | +1.3 | 0.75 | Gas concentration [ppm] | 307 | 230 | 62 | 599 |
| | | | | | | Current efficiency [%] | 26.8 | 30.0 | 9.6 | — |
| Example | 7 | 4 | 0.57 | +1.1 | 0.80 | Gas concentration [ppm] | 283 | 207 | 61 | 551 |
| 10 | | | | | | Current efficiency [%] | 24.7 | 27.0 | 9.4 | — |
| Example | 7 | 5 | 0.71 | +1.4 | 0.80 | Gas concentration [ppm] | 285 | 216 | 52 | 553 |
| 11 | | | | | | Current efficiency [%] | 24.9 | 28.2 | 8.1 | — |
| Example | 8 | 5 | 0.63 | +1.3 | 0.85 | Gas concentration [ppm] | 287 | 210 | 59 | 556 |
| 12 | | | | | | Current efficiency [%] | 25.1 | 27.4 | 9.1 | — |
| Example | 8 | 6 | 0.75 | +1.5 | 0.85 | Gas concentration [ppm] | 287 | 206 | 56 | 549 |
| 13 | | | | | | Current efficiency [%] | 25.1 | 26.9 | 8.7 | — |
| Example | 9 | 5 | 0.56 | +1.1 | 0.90 | Gas concentration [ppm] | 285 | 214 | 59 | 558 |
| 14 | | | | | | Current efficiency [%] | 24.9 | 28.0 | 9.1 | — |
| Example | 9 | 6 | 0.67 | +1.3 | 0.90 | Gas concentration [ppm] | 311 | 232 | 65 | 608 |
| 15 | | | | | | Current efficiency [%] | 27.2 | 30.3 | 10.1 | — |
| Example | 10 | 6 | 0.60 | +1.2 | 0.90 | Gas concentration [ppm] | 295 | 208 | 58 | 561 |
| 16 | | | | | | Current efficiency [%] | 25.8 | 27.2 | 9.0 | — |
| Example | 10 | 7 | 0.70 | +1.4 | 0.90 | Gas concentration [ppm] | 294 | 223 | 57 | 574 |
| 17 | | | | | | Current efficiency [%] | 25.7 | 29.1 | 8.8 | — |
| Example | 11 | 7 | 0.64 | +1.3 | 0.95 | Gas concentration [ppm] | 298 | 212 | 58 | 568 |
| 18 | | | | | | Current efficiency [%] | 26.1 | 27.7 | 9.0 | — |
| Example | 11 | 8 | 0.73 | +1.5 | 0.95 | Gas concentration [ppm] | 292 | 207 | 58 | 557 |
| 19 | | | | | | Current efficiency [%] | 25.5 | 27.0 | 9.0 | — |
| Example 4 | 12 | 0 | 0.00 | +0 | 0.90 | Gas concentration [ppm] | 197 | 159 | 35 | 391 |
| | | | | | | Current efficiency [%] | 17.2 | 20.8 | 5.4 | — |
| Example 3 | 12 | 6 | 0.50 | +1.0 | 0.90 | Gas concentration [ppm] | 267 | 204 | 58 | 529 |
| | | | | | | Current efficiency [%] | 23.3 | 26.7 | 9.0 | — |
| Example | 12 | 7 | 0.58 | +1.2 | 1.00 | Gas concentration [ppm] | 305 | 217 | 60 | 582 |
| 20 | | | | | | Current efficiency [%] | 26.7 | 28.4 | 9.3 | — |
| Example 1 | 12 | 8 | 0.67 | +1.3 | 1.00 | Gas concentration [ppm] | 326 | 240 | 68 | 634 |
| | | | | | | Current efficiency [%] | 28.5 | 31.4 | 10.5 | — |
| Example | 12 | 9 | 0.75 | +1.5 | 1.00 | Gas concentration [ppm] | 295 | 209 | 59 | 563 |
| 21 | | | | | | Current efficiency [%] | 25.8 | 27.3 | 9.1 | — |
| Example 2 | 12 | 12 | 1.00 | +2.0 | 1.20 | Gas concentration [ppm] | 251 | 193 | 54 | 498 |
| | | | | | | Current efficiency [%] | 21.9 | 25.2 | 8.4 | — |
| Example | 13 | 9 | 0.69 | +1.4 | 1.05 | Gas concentration [ppm] | 298 | 220 | 56 | 574 |
| 22 | | | | | | Current efficiency [%] | 26.1 | 28.7 | 8.7 | — |
| Example | 13 | 10 | 0.77 | +1.5 | 1.05 | Gas concentration [ppm] | 289 | 202 | 50 | 541 |
| 23 | | | | | | Current efficiency [%] | 25.3 | 26.4 | 7.7 | — |
| Example | 14 | 10 | 0.71 | +1.4 | 1.05 | Gas concentration [ppm] | 295 | 205 | 53 | 553 |
| 24 | | | | | | Current efficiency [%] | 25.8 | 26.8 | 8.2 | — |
| Example | 15 | 9 | 0.60 | +1.2 | 1.10 | Gas concentration [ppm] | 304 | 215 | 57 | 576 |
| 25 | | | | | | Current efficiency [%] | 26.6 | 28.1 | 8.8 | — |
| Example | 15 | 10 | 0.67 | +1.3 | 1.10 | Gas concentration [ppm] | 318 | 235 | 64 | 617 |
| 26 | | | | | | Current efficiency [%] | 27.8 | 30.7 | 9.9 | — |
| Example | 15 | 11 | 0.73 | +1.5 | 1.10 | Gas concentration [ppm] | 285 | 199 | 47 | 531 |
| 27 | | | | | | Current efficiency [%] | 24.9 | 26.0 | 7.3 | — |

As shown in Table 2, it was confirmed that the copper-containing cluster represented by CuₙOₘ exhibits particularly excellent catalytic activity when a ratio min is 0.67 (Examples 1, 6, 9, 15, and 26). It was confirmed that all the products can be generated in a largest amount when n is particularly 12 (Example 1) in the copper-containing cluster represented by CuₙOₘ with the ratio min of 0.67, and Example 1 has particularly excellent catalyst efficiency.

What is claimed is:

1. A metal-containing cluster catalyst comprising:
a cluster including at least one metal atom (M), a metal of each of the at least one metal atom (M) being selected from the group consisting of gold, silver, copper, platinum, rhodium, palladium, nickel, cobalt, iron, manganese, chromium, iridium, and ruthenium,
wherein the metal-containing cluster catalyst is used for reducing carbon dioxide.

2. The metal-containing cluster catalyst according to claim 1, wherein the cluster is composed of a metal oxide including the at least one metal atoms (M).

3. The metal-containing cluster catalyst according to claim 1, wherein the cluster is composed of a metal simple substance or metal oxide represented by the following general formula (1):
MₙOₘ (1),
where:
M represents the metal atom (M);
n and m are integers;
n is 30 or less; and
m is, with respect to n, such that a ratio m/n is 0 to 2.

4. The metal-containing cluster catalyst according to claim 3, wherein m is an integer, and with respect to n, such that m/n is 0.5 to 1.5.

5. The metal-containing cluster catalyst according to claim 1, wherein a primary particle diameter of the cluster is 0.1 to 3.0 nm.

6. An electrode for carbon dioxide reduction comprising:
a metal-containing cluster catalyst,
the metal-containing cluster catalyst including a cluster including at least one metal atom (M), a metal of each of the at least one metal atom (M) being selected from the group consisting of gold, silver, copper, platinum, rhodium, palladium, nickel, cobalt, iron, manganese, chromium, iridium, and ruthenium,
wherein the metal-containing cluster catalyst is used for reducing carbon dioxide.

7. A carbon dioxide reduction apparatus comprising:
an electrode for carbon dioxide reduction,
the electrode for carbon dioxide reduction including
a metal-containing cluster catalyst including a cluster including at least one metal atom (M), a metal of each of the at least one metal atom (M) being selected from the group consisting of gold, silver, copper, platinum, rhodium, palladium, nickel, cobalt, iron, manganese, chromium, iridium, and ruthenium,
wherein the metal-containing cluster catalyst is used for reducing carbon dioxide.
